(19) United States
(12) Patent Application Publication
(10) Pub. No.: US 2005/0089541 A1
(43) Pub. Date: Apr. 28, 2005

(54) Chlorhexidine-containing O/W emulsion

(75) Inventor: Stephane Lacoutiere, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/948,180

(22) Filed: Sep. 24, 2004

Related U.S. Application Data
(60) Provisional application No. 60/519,622, filed on Nov. 14, 2003.

(30) Foreign Application Priority Data
Oct. 23, 2003 (FR) 0350721

(51) Int. Cl. A61K 7/00

(52) U.S. Cl. 424/401

(57) ABSTRACT

The present invention relates to a composition in the form of an oil-in-water emulsion in which the mean size of the oil globules ranges from 50 nm to 1000 nm containing at least one chlorhexidine salt. The chlorhexidine salt may be in particular chlorhexidine digluconate. The emulsion has good bacteriological protection, and exhibits the advantage of having a pH which remains stable over time, even at a temperature above ambient temperature. The compositions of the invention can be used in numerous cosmetic and dermatological applications. They can also be used for impregnating a support and constituting a wipe for the skin.

### REFERENCE TO PRIOR APPLICATIONS

This application claims priority to U.S. provisional application 60/519,622 filed Nov. 14, 2003, and to French patent application 0350721 filed Oct. 23, 2003, both incorporated herein by reference.

### BRIEF DESCRIPTION OF THE INVENTION

The present invention relates in a preferred embodiment to a composition, preferably suitable for topical application, in the form of an oil-in-water emulsion comprising an oily phase dispersed in an aqueous phase, in which the mean size of the oil globules ranges from 50 nm to 1000 nm, wherein it comprises at least one chlorhexidine salt, the amount of chlorhexidine salt ranging from 0.05 to 3% by weight relative to the total weight of the composition, and wherein it comprises at least one emulsifier having an HLB of 9 to 18, selected from the group consisting of:
the compounds corresponding to formula (I):
   R—(O—CH₂—CH₂)ₙ—OH (I)
   in which R represents a saturated or unsaturated and linear or branched hydrocarbonaceous residue having from 8 to 30 carbon atoms and n represents a number ranging from 8 to 50, preferably from 8 to 30;
the addition products of 4 to 20 mol of ethylene oxide with one or more glycerol partial esters;
and their mixtures.

The present invention also relates in general to cosmetic and/or dermatological compositions in the form of an ultrafine oil-in-water emulsion, capable of being obtained by phase inversion, having stable pH and comprising a chlorhexidine salt.

The invention further relates to uses of the described compositions, in particular in the cosmetics field, as such or in the form of an article impregnated with the compositions.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

It is common to use, in the cosmetics field, emulsions, in particular emulsions of the oil-in-water type (oily phase dispersed in an aqueous phase constituting the continuous phase) which provide, during application to the skin, a soft and not very greasy feel.

Oil-in-water emulsions are generally stabilized by emulsifying surfactants of the oil-in-water type which, by virtue of their amphiphilic structure, become positioned at the oily phase/aqueous phase interface and thus stabilize the dispersed oil droplets. Despite the presence of emulsifiers, the emulsions may have a tendency to phase separate (separation of the aqueous and oily phases with release of oil). The increase in the content of emulsifier generally makes it possible to improve the stability of the emulsion. However, the presence of emulsifiers at high concentrations leads to problems of discomfort of the composition obtained when applied, such as, for example, a rough feel or a sticky or tacky feel, and to problems of harmlessness with respect to the skin, eyes and scalp, as a result of a large amount of emulsifiers.

The proposal has been made, to solve the problems of stability of conventional O/W emulsions, to prepare specific “ultrafine” O/W emulsions in which the mean size of the globules constituting the oily phase is within highly specific limits, namely between 50 and 1000 nm, the ultrafine emulsions of O/W type being themselves generally obtained according to a phase-inversion emulsification technique.

This technique is, in its principle, well known to a person skilled in the art and is described in particular in the articles “Phase Inversion Emulsification”, by T h. Förster et al., which appeared in Cosmetics & Toiletries, Vol. 106, December 1991, pp. 49-52; and “Application of the phase-inversion-temperature method to the emulsification of cosmetics” by T. Mitsui et al., which appeared in American Cosmetics and Perfumery, Vol. 87, December 1972. The principle of this technique, as currently described, is as follows: a W/O emulsion (introduction of the aqueous phase into the oily phase) is prepared at a temperature which has to be greater than the phase inversion temperature (PIT) of the system, that is to say the temperature at which the equilibrium between the hydrophilic and lipophilic properties of the emulsifier or emulsifiers employed is reached; at high temperature, that is to say greater than the phase inversion temperature (>PIT), the emulsion is of water-in-oil type and, during its cooling, this emulsion inverts at the phase inversion temperature to become an emulsion of oil-in-water type, this being achieved on being passed previously through a microemulsion state.

These PIT emulsions are most often very fluid. They have a bluish appearance and may be transluscent. However, these emulsions present problems of stability, and in particular of stability of the pH which tends to vary over time, in particular when the temperature exceeds ambient temperature (that is to say about 25° C.). In addition, their bacteriological protection also presents a problem because it is more difficult in conventional emulsions. However, it is important to have a composition whose pH remains stable, and moreover to have a composition which is well protected from a bacteriological point of view.

The need therefore remains for very fine O/W emulsions which have a pH which is stable over time, and which exhibit a good bacteriological protection.

### DETAILED DESCRIPTION OF THE PREFERRED EMBODIMENTS

The inventor has discovered, surprisingly, that the addition of a chlorhexidine salt makes it possible to obtain an O/W emulsion having not only good bacteriological protection, but also one having a stable pH over time.

The O/W emulsions of the present invention have the advantage of additionally being very well tolerated because chlorhexidine salts are preservatives and the addition of a small quantity of these preservatives, used in the composition according to the invention, is sufficient to obtain good bacteriological protection. Furthermore, they have the advantages of softness, freshness and comfort on application.

Indeed, the document “Physiochemical screening of antimicrobial agents as potential preservatives for submicron emulsions” by Sznitowska, Malgorzata et al., European Journal of Pharmaceutical Sciences (2002), 15(5), p. 489-495, describes the incorporation of chlorhexidine salts as preservatives in fine O/W emulsions obtained by high pressure homogenization (HPH), but the results described in this article show that the addition of such salts should be avoided because of problems of coalescence and of change in pH (page 493, right-hand column).

The emulsions according to the invention preferably contain particular emulsifying surfactants and have a defined quantity of chlorhexidine salts, which allows, unlike the prior art for the fine emulsions obtained by HPH, good stabilization of the pH.

One subject of the present invention is thus a composition, preferably suitable for topical application, in the form of an oil-in-water emulsion comprising an oily phase dispersed in an aqueous phase, in which the mean size of the oil globules ranges from 50 nm to 1000 nm (nanometres), wherein it comprises at least one chlorhexidine salt, the amount of chlorhexidine salt ranging from 0.05 to 3% by weight relative to the total weight of the composition, and in that it comprises at least one emulsifier having an HLB of 9 to 18, selected from the group consisting of:
the compounds corresponding to the formula (I):
   R—(O—CH₂—CH₂)ₙ—OH (I)
   in which R represents a saturated or unsaturated and linear or branched hydrocarbonaceous residue having from 8 to 30 carbon atoms and n represents a number ranging from 8 to 50, preferably from 8 to 30;
the addition products of 4 to 20 mol of ethylene oxide with one or more glycerol partial esters;
and their mixtures.

This composition can constitute in particular a cosmetic or dermatological composition.

The oil-in-water emulsion of the invention is in particular capable of being obtained according to the phase-inversion emulsification technique.

Owing to the fact that it is preferably intended for topical application, the composition of the invention may generally comprise a physiologically acceptable medium. The term “physiologically acceptable medium” is understood to mean a medium suitable for topical application to the skin or superficial body growths, that is to say compatible with the skin, mucous membranes, lips, hair and nails.

The emulsions according to the invention may be fluid to a greater or lesser degree. They are preferably generally fluid, and may have in this case a viscosity ranging, for example, from 1 to 500 cPoises (1 to 500 mPa.s) and preferably from 1 to 200 cPoises (1 to 200 mPa.s), the viscosity being measured at ambient temperature (25° C.) with a Rheomat RM 180 (generally with the No. 1 or 2 rotor). However, the emulsions according to the invention may also be less fluid.

As indicated above, the chlorhexidine salts used in the amounts described herein, and preferably in an amount of 0.1 to 2% of the total weight of the composition, make it possible to obtain compositions whose pH remains stable.

Accordingly, one subject of the present invention is also the use of at least one chlorhexidine salt, with an amount of chlorhexidine ranging from 0.05 to 3% by weight relative to the total weight of the composition, for stabilizing the pH of an oil-in-water emulsion in which the mean size of the oil globules ranges from 50 nm to 1000 nm.

According to a highly preferred characteristic of the compositions in accordance with the present invention, the number-average size of the liquid particles (or globules) of oil (or oily phase) within the dispersing aqueous phase ranges from 50 nm to 1000 nm. Preferably, this mean size ranges from 70 nm to 350 nm and more particularly from 70 nm to 300 nm.

In addition, the compositions according to the invention are preferably characterized by a very low polydispersity, that is to say that the size of the globules is very homogeneous. As a general rule, 90% of the oil globules of the ultrafine oil-in-water emulsion according to the invention have a number-average size ranging from 70 to 350 nm. The difference in size between the largest and smallest globules generally preferably ranges from 20 to 400 nm and more preferably from 30 to 200 nm, whereas, in conventional emulsions (other than PIT emulsions or other than microemulsions), the difference in size between the largest and smallest globules is generally greater than 1000 nm.

Useful chlorhexidine salts include, in particular, chlorhexidine digluconate, chlorhexidine diacetate and chlorhexidine hydrochloride. Chlorhexidine digluconate is preferably used. Of course, a mixture of these salts may be used.

The amount of chlorhexidine salt(s) in the composition of the invention may be that amount sufficient to produce the desired stability. This amount (by weight of active material) may for example range from 0.05 to 3% by weight, preferably from 0.1 to 2% by weight and even better from 0.15 to 1% by weight relative to the total weight of the composition.

The nature of the oily phase of the emulsions according to the invention is not critical. Thus, the oily phase can be composed of any compound(s) which is already generally known as being suitable for the manufacture of emulsions of oil-in-water type, in particular in the cosmetics field or dermatological field. In particular, these compounds can be chosen, alone or as mixtures, from oils of vegetable, animal or mineral origin, natural or synthetic waxes, and various other fatty substances, it being necessary for these oils, in particular cosmetic oils, and other fatty substances to be physiologically acceptable.

The oily phase usually comprises at least one oil. Useful oils which can participate in the composition of the oily phase include:
mineral oils, such as liquid paraffin and liquid petrolatum;
hydrocarbonaceous oils of animal origin, such as perhydrosqualene;
hydrocarbonaceous oils of vegetable origin, such as sweet almond oil, avocado oil, castor oil, coriander oil, olive oil, jojoba oil, sesame oil, groundnut oil, grape seed oil, rapeseed oil, coconut oil, hazelnut oil, karite butter, palm oil, apricot kernel oil, calophyllum oil, rice bran oil, maize germ oil, wheat germ oil, soybean oil, sunflower oil, evening primrose oil, safflower oil, passion flower oil, rye oil, or triglycerides of caprylic/capric acids, such as those sold by Stearineries Dubois or those sold under the names Miglyol 810, 812 and 818 by Dynamit Nobel;
synthetic oils, such as purcellin oil, fatty acid esters such as butyl myristate, isopropyl myristate, cetyl myristate, isopropyl palmitate, 2-ethylhexyl palmitate, isopropyl adipate, ethylhexyl adipate, butyl stearate, hexadecyl stearate, isopropyl stearate, octyl stearate, isocetyl stearate, decyl oleate, hexyl laurate, propylene glycol dicaprylate and the esters derived from lanolic acid, such as isopropyl lanolate or isocetyl lanolate, dioctylcyclohexane, isoparaffins and poly-α-olefins;
ethers, such as dicaprylyl ether (CTFA name: Dicaprylyl ether); benzoates of C₁₂-C₁₅ fatty alcohols (Finsolv TN from Finetex);
substantially linear or branched hydrocarbons of mineral or synthetic origin, such as volatile or nonvolatile paraffin oils, and their derivatives, petroleum jelly, polydecenes, isohexadecane, isododecane, hydrogenated polyisobutene such as Parleam® oil;
fatty alcohols comprising from 8 to 26 carbon atoms, such as lauryl alcohol, cetyl alcohol, myristyl alcohol, stearyl alcohol, palmityl alcohol, oleyl alcohol, linoleyl alcohol, 2-octyldodecanol, and their mixtures, such as cetearyl alcohol (mixture of cetyl alcohol and of stearyl alcohol);
acetylglycerides;
octanoates and decanoates of alcohols and of polyalcohols, such as those of glycol and of glycerol; ricinoleates of alcohols and of polyalcohols, such as those of cetyl;
fluorinated and perfluorinated oils;
lanolin; hydrogenated lanolin; acetylated lanolin;
volatile or nonvolatile silicone oils, such as volatile or nonvolatile polymethylsiloxanes (PDMS) with a linear or cyclic silicone chain which are liquid or pasty at ambient temperature, in particular cyclopolydimethylsiloxanes (cyclomethicones), such as cyclohexasiloxane; polydimethylsiloxanes comprising pendent alkyl, alkoxy or phenyl groups or alkyl, alkoxy or phenyl groups at the end of the silicone chain, which groups have from 2 to 24 carbon atoms; or phenylated silicones, such as phenyl trimethicones, phenyl dimethicones, phenyltrimethylsiloxydiphenylsiloxanes, diphenyl dimethicones, diphenylmethyl- diphenyltrisiloxanes, (2-phenylethyl)trimethylsiloxysilicates or polymethylphenylsiloxanes.

The term “hydrocarbonaceous” as used herein is understood to mean predominantly comprising carbon and hydrogen atoms and optionally ester, ether, fluorinated, carboxylic acid and/or alcohol groups.

Use may be made of one oil or a mixture of several of the oils indicated above.

According to a specific embodiment of the invention, the composition comprises at least one oil selected from mineral oils, fatty acid esters, and their mixtures.

According to another specific embodiment of the invention, the composition comprises at least one emollient selected from the oils indicated above and more particularly 2-ethylhexyl palmitate, dicaprylyl ether, and their mixtures.

The aqueous phase can comprise, in addition to water, one or more water-soluble solvents selected for example from polyols (or polyhydric alcohols), water-soluble lower alcohol(s), and their mixtures. The term “lower alcohol” is understood to mean an alcohol comprising from 1 to 8 carbon atoms. Mention may be made, as polyols, of, for example, glycerol; glycols, such as propylene glycol or butylene glycol; sorbitol; sugars, such as glucose, fructose, maltose, lactose or sucrose; and their mixtures. Mention may be made, as lower alcohols, of, for example, ethanol, isopropanol, butanol and their mixtures.

The oil-in-water emulsions in accordance with the invention preferably comprise:
(i) from 50 to 98% by weight, more preferably from 70 to 96% by weight, of aqueous phase with respect to the total weight of the composition;
(ii) from 2 to 50% by weight, more preferably from 4 to 30% by weight, of oily phase with respect to the total weight of the composition.

The emulsions in accordance with the invention generally comprise one or more emulsifying surfactants. In addition, they can comprise specific coemulsifiers, the role of which is, during the preparation of the emulsion, to substantially reduce the amount of emulsifying surface-active agents necessary for the preparation of the emulsion.

The composition of the invention preferably comprises at least one emulsifier having an HLB (“Hydrophilic Lipophilic Balance”) of 9 to 18, selected from the group consisting of:
the compounds corresponding to the formula (1):
   R—(O—CH₂—CH₂)ₙ—OH (I)
   in which R represents a saturated or unsaturated and linear or branched hydrocarbonaceous residue having from 8 to 30 carbon atoms and n represents a number ranging from 8 to 50, preferably from 8 to 30;
the addition products of 4 to 20 mol of ethylene oxide with one or more glycerol partial esters;
and their mixtures.

Use may be made, for example, as compounds of formula (I), of the addition products of ethylene oxide with behenyl alcohol, such as, for example, Beheneth-9 (9 mol of ethylene oxide) and Beheneth-10 (10 mol of ethylene oxide); those obtained with cetyl alcohol or with stearyl alcohol or with their mixtures, in particular those comprising 12 to 20 mol of ethylene oxide, such as, for example, those obtained from the mixture of cetyl alcohol and of stearyl alcohol (cetearyl alcohol), such as Ceteareth-12, Ceteareth-13, Ceteareth-14, Ceteareth-15, Ceteareth-16, Ceteareth-17, Ceteareth-18 or Ceteareth20; those obtained from cetyl alcohol, such as Ceteth-14, Ceteth-15, Ceteth-16 or Ceteth-20; those obtained from stearyl alcohol, such as Steareth-13, Steareth-14, Steareth-15, Steareth-16 or Steareth-20, and the mixtures of these various emulsifiers.

Use may also be made, as emulsifiers, of the addition products of 4 to 20 mol of ethylene oxide with one or more glycerol partial esters. The term “glycerol partial esters” is understood to mean, for example, the mixtures of mono-, di- and triglycerides of C₁₀-C₂₀ fatty acids obtained by esterification of one mole of glycerol by 1 or 2 mol of a C₁₀-C₂₀ fatty acid.

In addition, the emulsions according to the invention can furthermore comprise one or more coemulsifiers. This or these coemulsifiers can be chosen, for example, from C₆-C₂₂ fatty alcohols or esters of C₃-C₆ polyols with C₁₄-C₂₂ fatty acids, and their mixtures. Mention may be made, as coemulsifier, of, for example, cetyl alcohol, stearyl alcohol, cetearyl alcohol (mixture of cetyl alcohol and of stearyl alcohol), glyceryl stearate, and their mixtures.

Use may be made, for example, as mixture comprising emulsifier and coemulsifier, of the mixture sold under the name Emulgade SEV by Cognis, comprising Ceteareth-20, Ceteareth-12, hydrogenated palm oil and cetearyl alcohol.

The amount of emulsifiers and of coemulsifiers in the composition of the invention is not particularly limited and generally may range from 0.05 to 30% by weight, preferably from 0.5 to 20% by weight and better still from 1 to 10% by weight with respect to the total weight of the composition.

The composition according to the invention can also comprise any adjuvant or additive used in the fields under consideration and in particular in the cosmetics field or dermatological field.

Mention may in particular be made, among adjuvants capable of being present in the aqueous phase and/or in the oily phase of the emulsions in accordance with the invention (according to the water-soluble or fat-soluble nature of these adjuvants), of ionic or nonionic gelling agents and thickening agents; hydrophilic or lipophilic cosmetic or dermatological active principles; fragrances; preservatives; sequestering agents (EDTA); pigments; pearlescence agents; inorganic or organic fillers, such as talc, kaolin, silica powder or polyethylene powder; soluble dyes; sunscreen agents; basifying or acidifying agents; compounds improving the softness and slipperiness of the composition on application, such as cetyl dimethicones, and their mixtures. The amounts of these various adjuvants are those used in the field under consideration, for example from 0.01 to 20% of the total weight of the composition.

The choice of cosmetic and/or dermatological active principles which can be used in the composition of the invention depends on the purpose of the composition. Mention may be made, as active principles which can be used in the invention, of, for example, bactericides, such as octopirox, triclosan and triclocarban; keratolytic and anti-aging agents, such as hydroxy acids (α-hydroxy acids and β-hydroxy acids), for example lactic acid, glycolic acid, citric acid, salicylic acid and their derivatives; essential oils; vitamins and in particular retinol (vitamin A), ascorbic acid (vitamin C), tocopherol (vitamin E), niacinamide (vitamin PP or B3), panthenol (vitamin B5) and their derivatives (for example esters); coenzymes and in particular coenzyme Q10 or ubiquinone; enzymes, such as, for example, lipases, proteases, phospholipases, cellulases, peroxidases, in particular lactoperoxidases, catalases or superoxide dismutases, and plant extracts comprising the abovementioned enzymes; yeasts, such as Saccharomyces cerevisiae; steroids; antioxidants and agents for combating free radicals; moisturizing agents, such as polyols (glycerol, sorbitol, sugars), protein hydrolysates, urea and the mixtures comprising them; slimming agents, such as caffeine; antielastase and anticollagenase agents; agents for the treatment of greasy skin; and their mixtures.

Also, mention may in particular be made, as salicylic acid derivatives, of 5-(n-octanoyl)salicylic acid (CTFA name: Capryloyl Salicylic Acid), 5-(n-decanoyl)salicylic acid, 5-(n-dodecanoyl)salicylic acid, 5-(n-octyl)salicylic acid, 5-(n-heptyloxy)salicylic acid, 5-(tert-octyl)salicylic acid, 5-butoxysalicylic acid, 5-ethoxysalicylic acid, 5-methoxysalicylic acid, 5-propoxysalicylic acid, 5-methylsalicylic acid, 5-ethylsalicylic acid, 5-propylsalicylic acid, and their mixtures. These acids can optionally be salified by a base.

Also, mention may be made, as examples of steroids, of, for example, dehydroepiandrosterone (or DHEA), as well as (1) its biological derivatives and precursors, in particular DHEA salts and esters, such as DHEA sulphate and salicylate, 7-hydroxy-DHEA, 7-keto-DHEA, or esters of 7-hydroxy- and 7-keto-DHEA, in particular 3-β-acetoxy-7-oxo-DHEA, and (2) its chemical derivatives and precursors, in particular sapogenins, such as diosgenin or hecogenin, and/or their derivatives, such as hecogenin acetate, and/or the natural extracts comprising them and in particular extracts of Dioscorea species, such as wild yam.

The sunscreen agents (or UV screening agents) optionally present in the composition can be selected from, e.g., organic screening agents, physical screening agents and their mixtures.

The composition of the invention can comprise, as chemical sunscreen agents which can be used in the composition of the invention, any UV-A and UV-B screening agent, including for example those which can be used in the cosmetics field.

Useful UV-B screening agents include, for example:
(1) salicylic acid derivatives, in particular homomenthyl salicylate and octyl salicylate;
(2) cinnamic acid derivatives, in particular 2-ethylhexyl p-methoxycinnamate, sold by Givaudan under the name Parsol MCX;
(3) liquid β,β′-diphenylacrylate derivatives, in particular 2-ethylhexyl α-cyano-β,β′-diphenylacrylate or octocrylene, sold by BASF under the name Uvinul N539;
(4) p-aminobenzoic acid derivatives;
(5) 4-methylbenzylidenecamphor, sold by Merck under the name Eusolex 6300;
(6) 2-phenylbenzimidazole-5-sulphonic acid, sold under the name Eusolex 232 by Merck;
(7) 1,3,5-triazine derivatives, in particular:
2,4,6-tris[p-(2′-ethylhexyl-1′-oxycarbonyl)anilino]-1,3,5-triazine, sold by BASF under the name Uvinul T150, and
dioctylbutamidotriazone, sold by Sigma 3V under the name Uvasorb HEB;
(8) the mixtures of these screening agents.

Useful UV-A screening agents include, for example:
(1) dibenzoylmethane derivatives, in particular 4-(tert-butyl)-4′-methoxydibenzoylmethane, sold by Givaudan under the name Parsol 1789;
(2) benzene- 1,4-[di(3-methylidenecamphor-10-sulphonic)]acid, optionally in the partially or completely neutralized form, sold under the name Mexoryl SX by Chimex;
(3) benzophenone derivatives, for example:
2,4-dihydroxybenzophenone (benzophenone-1);
2,2′,4,4′-tetrahydroxybenzophenone (benzophenone-2);
2-hydroxy-4-methoxybenzophenone (benzophenone-3), sold under the name Uvinul M40 by BASF;
2-hydroxy-4-methoxybenzophenone-5-sulphonic acid (benzophenone-4) and its sulphonate form (benzophenone-5), sold by BASF under the name Uvinul MS40;
2,2′-dihydroxy-4,4′-dimethoxybenzophenone (benzophenone-6);
5-chloro-2-hydroxybenzophenone (benzophenone-7);
2,2′-dihydroxy-4-methoxybenzophenone (benzophenone-8);
the disodium salt of 2,2′-dihydroxy-4,4′-dimethoxybenzophenone-5,5′-disulphonic diacid (benzophenone-9);
2-hydroxy-4-methoxy-4′-methylbenzophenone (benzophenone-10);
benzophenone-11;
2-hydroxy-4-(octyloxy)benzophenone (benzophenone-12);
(4) silane derivatives or polyorganosiloxanes comprising a benzophenone group;
(5) anthranilates, in particular menthyl anthranilate, sold by Haarmann & Reimer under the name Neo Heliopan M A;
(6) compounds comprising, per molecule, at least two benzazolyl groups or at least one benzodiazolyl group, in particular 1,4-bis(benzimidazolyl)phenylene-3,3′, 5,5′-tetrasulphonic acid and its salts, sold by Haarmann & Reimer;
(7) silicon derivatives of N-substituted benzimidazolylbenzazoles or of benzofuranyl-benzazoles, and in particular:
2-[1-[3-[1,3,3,3-tetramethyl-1-[(trimethylsilyl)oxy]disiloxanyl]propyl]-1H-benzimidazol-2-yl]benzoxazole;
2-[1-[3-[1,3,3,3-tetramethyl-1-[(trimethylsilyl)oxy]disiloxanyl]propyl]-1H-benzimidazol-2-yl]benzothiazole;
2-[1-(3-(trimethylsilanyl)propyl)-1H-benzimidazol-2-yl]benzoxazole;
6-methoxy-1,1′-bis(3-(trimethylsilanyl)propyl)-1H, 1′H-[2,2′]bibenzimidazolylbenzoxazole;
2-[1-(3-(trimethylsilanyl)propyl)-1H-benzimidazol-2-yl]benzothiazole; which are disclosed in Patent Application EP-A-1 028 120;
(8) triazine derivatives and in particular 2,4-bis[4-(2-ethylhexyloxy)-2-hydroxyphenyl]-6-(4-methoxyphenyl)-1,3,5-triazine, sold by Ciba Geigy under the name Tinosorb S, and 2,2′-methylenebis[6-(2H-benzotriazol-2-yl)-4-(1,1,3,3-tetramethylbutyl) phenol], sold by Ciba Geigy under the name Tinosorb M;
(9) their mixtures.

It is also possible to use a mixture of several of these screening agents and a mixture of UV-B screening agents and of UV-A screening agents and also mixtures with physical screening agents.

Useful physical screening agents include titanium oxide (amorphous titanium dioxide or crystalline titanium dioxide in the rutile and/or anatase form), zinc oxide, iron oxide, zirconium oxide, cerium oxide or their mixtures. These metal oxides can be in the form of particles having a micrometric size or nanometric size (nanopigments). In the form of nanopigments, the mean sizes of the particles range, for example, from 5 to 100 nm. Nanopigments are preferably used in the composition of the invention.

Of course, a person skilled in the art will take care to choose the possible compound or compounds to be added to the composition according to the invention so that the advantageous properties intrinsically attached to the composition in accordance with the invention are not, or not substantially, detrimentally affected by the addition.

The emulsions according to the invention may be obtained by a phase inversion process. This preparation process is one wherein:
1) (A) the oily phase, this oily phase comprising at least one oil and optionally other oils and fatty substances and any fat-soluble adjuvants which are stable at the phase inversion temperature,
(B) at least one emulsifier and optionally a coemulsifier,
(D) the aqueous phase, this aqueous phase comprising water and optionally the water-soluble solvents and the water-soluble adjuvants which are stable at the phase inversion temperature,
are mixed in order to produce a conventional emulsion,
(2) this emulsion is heated to a temperature situated within or above the phase inversion region, or else the emulsion is prepared directly at such a temperature, then
(3) the emulsion is cooled and the chlorhexidine salt and optionally any adjuvants or active principles which are unstable at the phase inversion temperature, such as, for example, fragrances, are added.

It is also possible to use other procedures, and for example to add only part of the aqueous phase during the preparation of the emulsion (for example 10 to 30% of the aqueous phase) and to then dilute the emulsion with the remainder of the aqueous phase containing the chlorhexidine salt(s) after cooling.

The phase inversion temperature region may be observed visually by observing the changes in transparency and opacity of the emulsion, the emulsion being opaque when it is initially in the form of a W/O emulsion, and then transparent when it becomes a microemulsion, again opaque when it is in the form of an O/W emulsion, and then again transparent when it is cooled below the phase inversion temperature. This temperature region may also be established for a given composition by measuring the conductivity of a sample of the composition which is heated. When the phase inversion region is reached, the conductivity of the emulsion increases very rapidly: in the phase inversion region, an increase in the conductivity of approximately 50 microsiemens per centimetre may be observed over a temperature range of 5 to 15° C., whereas it would only be approximately 5 microsiemens per centimetre over an equivalent temperature range outside the phase inversion region.

The compositions according to the invention can be provided in any form, and in particular in the form of a lotion or a milk which is more or less fluid. They can be used as such or as an aerosol or in the form of a spray.

The compositions of the invention can constitute in particular care, protection and/or hygiene products for the skin of the face and/or of the body, the scalp or mucous membranes, such as protective, treatment or care sprays for the face, for the hands or for the body, or body milks or lotions for protection or care of the skin, scalp or mucous membranes or for removing make-up from and/or cleaning the skin and/or lips. They can be used, for example, for moisturizing and/or firming up the skin and/or for nourishing the skin.

The compositions according to the invention can also constitute antisun products, for protecting the skin of the body and/or of the face from the harmful effects of the sun or UV radiation, and also products for making up keratinous substances and in particular the skin and/or lips.

One subject of the present invention is thus the cosmetic use of the composition as defined above for treating, protecting, caring for, removing make-up from and/or cleaning the skin and/or lips and/or for making up the skin and/or lips.

Another subject of the present invention is also a process for the cosmetic treatment of the skin and/or the lips, wherein a composition as defined above is applied to the skin and/or the lips. The type of treatment can vary in particular as a function of the active principle or principles present in the composition.

According to another embodiment of the invention, the compositions according to the invention can be used for impregnating a water-insoluble substrate in order to form an article to be applied to the skin, such as a wipe. The water-insoluble substrate can be selected from the non-limiting group consisting of woven materials, nonwoven materials, foams, sponges and waddings, as sheets, balls or films. It can in particular be a nonwoven substrate based on fibres of natural origin (linen, wool, cotton, silk) or of synthetic origin (cellulose derivatives, viscose, polyvinyl derivatives, polyesters, such as poly(ethylene) terephthalate, polyolefins, such as polyethylene or polypropylene, polyamides, such as Nylon, or acrylic derivatives). Nonwovens are described in a general way in Riedel, “Nonwoven Bonding Methods & Materials”, Nonwoven World (1987).

These substrates are obtained according to conventional processes in the art of preparing nonwovens. According to a specific embodiment of the invention, the insoluble substrate can comprise at least one of the compounds and in particular preservatives of the invention, attached to the support by known means for grafting biocidal agents to fibres.

This substrate can comprise one or more layers having identical or different properties and can have properties of elasticity and of softness and other properties appropriate for the desired use. The substrates can comprise, for example, two parts having different properties of elasticity, as disclosed in the document WO-A-99/13861, or can comprise a single layer with different densities, as disclosed in the document WO-A-99/253 18, or can comprise two layers of different textures, as disclosed in the document WO-A-98/18441.

The substrate can have any size and any shape appropriate for the desired purpose. It generally preferably has a surface area of between 0.005 m² and 0.1 m², preferably between 0.01 m² and 0.05 m². It is preferably provided in the form of rectangular wipes or of round compresses.

The final article comprising the substrate and the impregnation composition is generally in the wet state, with a degree of impregnation of the composition ranging, for example, from 200 to 1000% and preferably from 250 to 350% by weight of composition with respect to the weight of substrate. Techniques for impregnating substrates with compositions are well known in this field and are all applicable to the present invention. In general, the impregnation composition is added to the substrate by one or more techniques comprising immersion, coating, vaporization, and the like.

It is also possible to form an article (or wipe) presented in the dry state, either by removing the water from the composition after its impregnation on the substrate or by impregnating the substrate with a composition in dry form in the powder, granule or film state by any known implementation means, such as welding and bonding of multilayers by thermal or ultrasound routes. In the latter embodiment, the composition is dried by any known means: atomization, lyophilization or other analogous process.

It is thus possible to obtain, according to the use envisaged, wet wipes or dry Wipes. The wet wipes can be used as such, whereas the dry wipes may be wetted before use.

Another subject of the invention is thus an article comprising (A) a water-insoluble substrate, (B) a composition as defined above, added to or impregnated on the substrate, and the uses of the article in the cosmetics or dermatological field, in particular for caring for (nourishing, firming up, moisturizing), cleaning and/or removing make-up from the skin.

This article can constitute in particular wipes for caring for and/or treating the skin and/or wipes for cleaning and/or removing make-up from the skin and/or eyes.

Another subject of the invention is the cosmetic use of an article as defined above, for caring for, cleaning and/or removing make-up from the skin and/or eyes.

The examples indicated below Will make possible a better understanding of the invention without, however, exhibiting a limiting nature. The amounts shown are as % by weight, unless otherwise mentioned. The names are chemical names and CTFA names, according to the compounds.

### EXAMPLE 1

### (Invention): Fluid Milk

| | |
|---|---|
| Phase A | |
| Mineral oil | 5% |
| Dicaprylyl ether (Cetiol OE from Cognis) | 3% |
| Mixture of Ceteareth-20, Ceteareth-12, | 3% |
| hydrogenated palm oil and cetearyl alcohol | |
| (Emulgade SEV from Cognis) | |
| Ceteareth-20 (Emulgin B2 from Cognis) | 1% |
| 2-Ethylhexyl palmitate | 3% |
| Cetyldimethicone (Abil Wax 9801 from | 0.1% |
| Goldschmidt) | |
| Phase B | |
| Glycerol | 5% |
| Distilled water | 15% |
| Phase C | |
| Palm oil | 0.2% |
| Fragrance | 0.4% |
| Phase D | |
| Chlorhexidine digluconate | 0.25% |
| Distilled water | q.s. for 100% |

Procedure: Phases A and B were heated separately, with stirring, and were homogenized. Phase B was poured into phase A while stirring slowly, and the mixture was heated as far as the phase inversion temperature (PIT) which is at a temperature of about 60 to 80° C. The water-in-oil emulsion obtained becomes virtually transparent during the heating. The heating is stopped when the emulsion becomes white and the phase C was poured into the emulsion while maintaining slow stirring, and then as soon as the emulsion becomes transparent during cooling, it was diluted by adding phase D.

A slightly bluish, fluid (viscosity of about 1 cPoise) oil-in-water emulsion was obtained. After storing at various temperatures, the pH remains stable as shown in the table below:

| Storage for two months at | Variation of pH |
|---|---|
| Ambient temperature | +0.15 |
| (about 25° C.) | |
| 37° C. | +0.15 |
| 45° C. | +0.05 |

In addition, microbial protection was good.

This emulsion can for example be used as such or as a spray or impregnated into a nonwoven support to constitute a wipe to be applied to the face or the body, for example for nourishing, moisturizing and firming the skin.

### EXAMPLE 2

### Comparative

| | |
|---|---|
| Phase A | |
| Mineral oil | 5% |
| Dicaprylyl ether (Cetiol OE from Cognis) | 3% |
| Mixture of Ceteareth-20, Ceteareth-12, | 3% |
| hydrogenated palm oil and cetearyl alcohol | |
| (Emulgade SEV from Cognis) | |
| Ceteareth-20 (Emulgin B2 from Cognis) | 1% |
| 2-Ethylhexyl palmitate | 3% |
| Cetyldimethicone (Abil Wax 9801 from | 0.1% |
| Goldschmidt) | |
| Phase B | |
| Glycerol | 5% |
| Distilled water | 15% |
| Methylparaben | 0.2% |
| Phase C | |
| Palm oil | 0.2% |
| Fragrance | 0.4% |
| Phase D | |
| Imidazolidinylurea (Germall 115) | 0.3% |
| Distilled water | q.s. for 100% |

The procedure was identical to that of Example 1 according to the invention.

In this example, chlorhexidine digluconate was replaced with another preservative (Germall 115).

It was observed that the pH varied over time as shown in the table below:

| Storage for two months at | Variation of pH |
|---|---|
| Ambient temperature | −0.57 |
| (about 25° C.) | |
| 37° C. | −1.11 |
| 45° C. | −1.28 |

In addition, microbial protection was found to be insufficient.

### EXAMPLE 3

### Comparative

| | |
|---|---|
| Phase A | |
| Mineral oil | 5% |
| Dicaprylyl ether (Cetiol OE from Cognis) | 3% |
| Mixture of Ceteareth-20, Ceteareth-12, | 3% |
| hydrogenated palm oil and cetearyl alcohol | |
| (Emulgade SEV from Cognis) | |
| Ceteareth-20 (Emulgin B2 from Cognis) | 1% |
| 2-Ethylhexyl palmitate | 3% |
| Cetyldimethicone (Abil Wax 9801 from | 0.1% |
| Goldschmidt) | |
| Phase B | |
| Glycerol | 5% |
| Distilled water | 15% |
| Methylparaben | 0.2% |
| Phase C | |
| Palm oil | 0.2% |
| Fragrance | 0.4% |
| Phase D | |
| Chlorphenesin | 0.25% |
| Distilled water | q.s. for 100% |

The procedure was identical to that of Example 1 according to the invention.

In this example, the chlorhexidine digluconate of Example 1 was replaced with another preservative (chlorphenesin).

It was observed that the pH varied over time as shown in the table below:

| Storage for two months at | Variation of pH |
|---|---|
| Ambient temperature | −0.24 |
| (about 25° C.) | |
| 37° C. | −0.22 |
| 45° C. | −0.65 |

In addition, microbial protection was insufficient.

### EXAMPLE 4

### (Invention): Fluid Milk

| | |
|---|---|
| Phase A | |
| Cyclomethicone | 4.5% |
| Dicaprylyl ether (Cetiol OE from Cognis) | 3% |
| Mixture of Ceteareth-20, Ceteareth-12, | 3% |
| hydrogenated palm oil and cetearyl alcohol | |
| (Emulgade SEV from Cognis) | |
| Ceteareth-15 (Mergital CS15 from Cognis) | 1.5% |
| Glyceryl stearate | 0.9% |
| Fragrance | 0.7% |
| Phase B | |
| Glycerol | 5% |
| Distilled water | 15% |
| Methylparaben | 0.2% |
| Phase C | |
| Ethyl alcohol | 3% |
| Phase D | |
| Chlorhexidine digluconate | 0.25% |
| Distilled water | q.s. for 100% |

The procedure was the same as for Example 1.

A slightly bluish, fluid (viscosity of about 1 cPoise) oil-in-water emulsion was obtained. After storing at various temperatures, the pH remained stable as shown in the table below:

| Storage for two months at | Variation of pH |
|---|---|
| Ambient temperature | +0.05 |
| (about 25° C.) | |
| 37° C. | +0.06 |
| 45° C. | +0.04 |

In addition, microbial protection was good.

This emulsion can for example be used as such or as a spray or impregnated into a nonwoven support to constitute a wipe to be applied to the face or the body, and for example used for nourishing, moisturizing and firming the skin.

The above written description of the invention provides a manner and process of making and using it such that any person skilled in this art is enabled to make and use the same, this enablement being provided in particular for the subject matter of the appended claims, which make up a part of the original description. In this regard, the above disclosure fully describes and enables a composition suitable for topical application in the form of an oil-in-water emulsion, comprising an oily phase dispersed in an aqueous phase, wherein the mean size of oil globules is from 50 nm to 1000 nm, at least one chlorhexidine salt in an amount of from 0.05 to 3% by weight relative to the total weight of the composition, and at least one emulsifier having an HLB of 9 to 18 and selected from the group consisting of:
compounds corresponding to formula (I):
   R—(O—CH₂—CH₂)ₙ—OH (I)
   in which R represents a saturated or unsaturated, linear or branched hydrocarbon group having from 8 to 30 carbon atoms and optionally comprising one or more ester, ether, fluorine, carboxylic acid and/or alcohol groups, and n represents a number ranging from 8 to 50;
the addition products of 4 to 20 mol of ethylene oxide with one or more glycerol partial esters;
and mixtures thereof.

As used above, the phrases “selected from the group consisting of,”“chosen from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition suitable for topical application in the form of an oil-in-water emulsion, comprising
an oily phase dispersed in an aqueous phase, wherein the mean size of oil globules is from 50 nm to 1000 nm,
at least one chlorhexidine salt in an amount of from 0.05 to 3% by weight relative to the total weight of the composition, and
at least one emulsifier having an HLB of 9 to 18 and selected from the group consisting of:
compounds corresponding to formula (I):
R—(O—CH₂—CH₂)ₙ—OH (I)
in which R represents a saturated or unsaturated, linear or branched hydrocarbon group having from 8 to 30 carbon atoms and optionally comprising one or more ester, ether, fluorine, carboxylic acid and/or alcohol groups, and n represents a number ranging from 8 to 50;
the addition products of 4 to 20 mol of ethylene oxide with one or more glycerol partial esters;
and mixtures thereof:

2. A composition according to claim 1, prepared by a phase-inversion emulsification technique.

3. A composition according to claim 1, wherein the chlorhexidine salt is selected from chlorhexidine digluconate, chlorhexidine diacetate, chlorhexidine hydrochloride, and mixtures thereof.

4. A composition according to claim 1, wherein the amount of chlorhexidine salt is from 0.1 to 2% by weight relative to the total weight of the composition.

5. A composition according to claim 1, wherein the oily phase comprises at least one oil selected from the group consisting of mineral oils, fatty acid esters and mixtures thereof.

6. A composition according to claim 1, wherein the aqueous phase represents from 50 to 98% by weight relative to the total weight of the composition.

7. A composition according to claim 1, wherein the emulsifier is selected from the group consisting of products of addition of ethylene oxide with behenyl alcohol, cetyl alcohol, stearyl alcohol, and mixtures thereof

8. A composition according to claim 1, further comprising a coemulsfier selected from the group consisting of C₁₆-C₂₂ fatty alcohols, partial esters of C₃-C₆ polyols with C₁₄-C₂₂ fatty acids, and mixtures thereof

9. A composition according to claim 8, wherein the amount of emulsifiers and of coemulsifiers ranges from 0.05 to 30% by weight with respect to the total weight of the composition.

10. A composition according to claim 1, wherein said composition constitutes a cosmetic or dermatological composition.

11. A composition according to claim 1, wherein said composition wherein it constitutes a care, protection and/or hygiene product for the skin of the face and/or of the body, the scalp or mucous membranes.

12. A method for treating, protecting, caring for, removing make-up from and/or cleaning the skin and/or lips, and/or for making up the skin and/or the lips and/or as sunscreen product, comprising applying to the skin and/or the lips the composition of claim 1.

13. A method for the cosmetic treatment of the skin and/or lips, comprising applying to the skin and/or the lips the composition of claim 1.

14. A method, comprising impregnating a water-insoluble substrate with the composition of claim 1.

15. An article comprising:
(A) a water-insoluble substrate, and
(B) a composition according to claim 1, added to or impregnated on the substrate.

16. An article according to claim 15, wherein the water-insoluble substrate is selected from the group consisting of woven materials, nonwoven materials, foams, sponges, waddings and combinations thereof.

17. An article according to claims 15, wherein said article constitutes a wipe for caring for and/or treating the skin or a wipe for cleaning and/or removing make-up from the skin and/or eyes.

18. An article according to claims 16, wherein said article constitutes a wipe for caring for and/or treating the skin or a wipe for cleaning and/or removing make-up from the skin and/or eyes.

19. A method for stabilizing the pH of an oil-in-water emulsion in which the mean size of oil globules ranges from 50 nm to 1000 nm, comprising adding to said emulsion at least one chlorhexidine salt in an amount of from 0.05 to 3% by weight relative to the total weight of the composition.

20. A method for preparing a pH stable oil-in-water emulsion wherein the mean size of oil globules ranges from 50 nm to 1000 nm, comprising combining in said emulsion during the preparation thereof at least one chlorhexidine salt in an amount of from 0.05 to 3% by weight relative to the total weight of the composition.
